(19) United States
(12) Patent Application Publication
(10) Pub. No.: US 2023/0074363 A1
(43) Pub. Date: Mar. 9, 2023

(54) METHODS AND SYSTEMS FOR MACHINE LEARNING-POWERED IDENTIFICATION OF CANCER DIAGNOSES AND DIAGNOSIS DATES FROM ELECTRONIC HEALTH RECORDS

(71) Applicant: Sema4 OpCo, Inc., Stamford, CT (US)

(72) Inventors: Yunchen Yang, Stamford, CT (US); Timmy O'Connell, Stamford, CT (US); Xiang Zhou, Stamford, CT (US); David Corrigan, Stamford, CT (US); Rong Chen, Stamford, CT (US)

(21) Appl. No.: 17/822,206

(22) Filed: Aug. 25, 2022

Related U.S. Application Data
(60) Provisional application No. 63/260,565, filed on Aug. 25, 2021.

(51) Int. Cl. G16H 10/60; G16H 50/20

(52) U.S. Cl. CPC G16H 10/60; G16H 50/20; G16H 50/70

(57) ABSTRACT

A method (100) for diagnosing a subject with cancer using a cancer diagnosis system (200), comprising: receiving (120), from an electronic health record database, a plurality of medical records for a subject; analyzing (130), by a trained cancer diagnosis model (263) of the system, the received plurality of medical records for the subject; generating (140), by the analysis, a cancer diagnosis for the subject, wherein the cancer diagnosis comprises both an identification of a cancer type and a date of diagnosis; and providing (150), via a user interface (240) of the system, the generated cancer diagnosis.

### CROSS-REFERENCE TO RELATED APPLICATIONS

This application claims priority to U.S. Provisional Patent Application Ser. No. 63/260,565, entitled “Methods and Systems for Machine Learning-Powered Identification of Cancer Diagnoses and Diagnosis Dates from Electronic Health Records”, filed on Aug. 25, 2021, which application is hereby incorporated by reference in its entirety.

### FIELD OF THE DISCLOSURE

The present disclosure is directed generally to methods and systems for identifying cancer diagnosis from electronic health records using a cancer diagnosis analysis system.

### BACKGROUND

Cancer is the leading cause of death worldwide, with 10 million deaths recorded in 2020. Although various therapeutic breakthroughs have occurred in the past several decades, more extensive research leveraging the data of large cohorts of cancer patients could further decipher the complexity of the disease and improve the therapeutic landscape of cancer. Among the studies of cancer research, the accurate identification of cancer patients and the extraction of diagnosis-related temporal information is quite crucial, which is an essential first step of numerous down-streaming analysis of precision oncology, such as epidemiology, drug development and biomarker discovery.

Electronic health record (EHR) documents contain rich information regarding patient health, diagnosis, testing and treatment. The EHR data therefore holds great potential in facilitating various aspects of clinical or translational research, especially cancer research, including case identification, disease information extraction and clinical decision support. However, EHR data is also sparse and noisy, where nuanced information is mostly embedded in the narrative text. Traditionally, chart review on EHR data is tedious and time-consuming, which cannot adapt to big data analysis.

To detect cases of diseases using EHR data, recent advances typically applied machine learning (ML) approaches to EHR data for various classification and prediction tasks. The features utilized by the ML models included both structured coded information (e.g., laboratory results, International Classification of Diseases (ICD) codes, demographic information, medication table, etc.) and unstructured narrative information extracted from free context of medical notes. At least one study trained a supporting vector machine (SVM) model with structured data, including lab results and vital signs, to classify 10 different cancer types, having achieved overall 86.3% accuracy. However, this study used ICD codes as gold standard of cancer diagnosis, which usually contains many false positive cases, compromising the accuracy of the performance evaluation. Another study attempted to utilize structured data elements only as features to train a random forest (RF)-based classifier to identify metastatic prostate cancer cases. Instead of using all available records, a time-windowing method was used to focus on information within specific temporal range. The approach eventually accomplished precision of 0.9 and recall of 0.4. Despite the high precision, the low recall revealed that the structured data elements alone are not comprehensive enough for disease case identification, where many true positive cases were missed. Compared with structured data, the unstructured content of EHR is more relevant and richly detailed, which therefore plays more significant role in disease identification.

Despite the advances of case detection from EHR data, challenges still remain. For some of the previous works, the ICD coded diagnosis was used as a standard of diagnosis. However, according to recent studies and our observations, ICD coded diagnosis may contain up to 50% false positive cases. To conduct effective and reproducible case detection, a standardized abstraction criteria and an accurately annotated gold standard dataset are required. Moreover, the prior studies usually focused on the case detection of a few specific cancer types, but none of them integrated such analysis across all the common cancer types. The integrated analysis can not only provide a high-dimensional patient-centered diagnosis information but also benefit various down-streaming translational studies of cancer research. In addition, to our best knowledge, the extraction of temporal information of diagnosis is rarely covered in current studies. In cancer research, nonetheless, the date of diagnosis (DDx) is a critical information element which is necessary for preventive surveillance, treatment evaluation and survival analysis.

### SUMMARY OF THE DISCLOSURE

Accordingly, there is a continued need for methods and systems capable of efficiently analyzing EHR data to more accurately diagnose a subject with cancer. Various embodiments and implementations herein are directed to a trained cancer diagnosis model and system configured to diagnose a subject with cancer using EHR data for that subject. A cancer diagnosis system receives a plurality of medical records for the subject from an electronic health record database. The system uses a trained cancer diagnosis model to analyze the received plurality of medical records for the subject. The trained cancer diagnosis model is trained by: (i) providing a curated cancer dictionary, the curated cancer dictionary comprising a plurality of cancer-related terms each associated with one or more types of cancer, wherein each of the plurality of terms in the curated cancer dictionary is associated with one or more of the following plurality of diagnosis categories: histology, diagnosis test method, stage, grade, and invasiveness; (ii) receiving a training dataset, comprising a plurality of medical records for each of a plurality of subjects; (iii) parsing (340), using the curated cancer dictionary and a natural language processing (NLP) algorithm, the training dataset to identify cancer-related terms in the plurality of medical records, wherein each of the identified cancer-related terms is associated with one or more of the plurality of diagnosis categories; (iv) analyzing, using a classifier, the training dataset to identify a cancer diagnosis date for each of the plurality of subjects; (v) generating a table of parsed cancer-related terms and cancer diagnosis date for each of the plurality of subjects; (vi) training, using the generated tables, a cancer diagnosis model to determine a cancer diagnosis and a cancer diagnosis date for a subject using a plurality of medical health records for that subject; and (vii) storing the trained cancer diagnosis model. Based on the analysis, the trained cancer diagnosis model generates a cancer diagnosis for the subject, wherein the cancer diagnosis comprises both an identification of a cancer type and a date of diagnosis. The system then provides the generated cancer diagnosis to a user or clinician via a user interface. According to an embodiment, the method further includes determining, based on the generated cancer diagnosis, a cancer-specific treatment for the subject. The clinician can then administer the cancer-specific treatment to the subject, wherein the cancer-specific treatment is one or more of radiation therapy, chemotherapy, immunotherapy, and surgery.

Generally, in one aspect, a method for diagnosing a subject with cancer using a cancer diagnosis system is provided. The method includes: receiving, from an electronic health record database, a plurality of medical records for a subject; analyzing, by a trained cancer diagnosis model of the system, the received plurality of medical records for the subject, wherein the cancer diagnosis model is trained by: (i) providing a curated cancer dictionary, the curated cancer dictionary comprising a plurality of cancer-related terms each associated with one or more types of cancer, wherein each of the plurality of terms in the curated cancer dictionary is associated with one or more of the following plurality of diagnosis categories: histology, diagnosis test method, stage, grade, and invasiveness; (ii) receiving a training dataset, comprising a plurality of medical records for each of a plurality of subjects; (iii) parsing, using the curated cancer dictionary and a natural language processing (NLP) algorithm, the training dataset to identify cancer-related terms in the plurality of medical records, wherein each of the identified cancer-related terms is associated with one or more of the plurality of diagnosis categories; (iv) analyzing, using a classifier, the training dataset to identify a cancer diagnosis date for each of the plurality of subjects; (v) generating a table of parsed cancer-related terms and cancer diagnosis date for each of the plurality of subjects; (vi) training, using the generated tables, a cancer diagnosis model to determine a cancer diagnosis and a cancer diagnosis date for a subject using a plurality of medical health records for that subject; and (vii) storing the trained cancer diagnosis model; generating, by the analysis, a cancer diagnosis for the subject, wherein the cancer diagnosis comprises both an identification of a cancer type and a date of diagnosis; and providing, via a user interface of the system, the generated cancer diagnosis.

According to an embodiment, the method further includes determining, based on the generated cancer diagnosis, a cancer-specific treatment for the subject; and administering the cancer-specific treatment to the subject, wherein the cancer-specific treatment is one or more of radiation therapy, chemotherapy, immunotherapy, and surgery.

According to an embodiment, the curated cancer dictionary is generated by: (i) receiving a plurality of medical records for a plurality of patients, wherein the plurality of patients may be a randomly selected subset of a larger plurality of patients; (ii) manually reviewing, by a clinician, the plurality of medical records for each of the plurality of patients, wherein manually reviewing by the clinician comprises annotating the plurality of medical records with a diagnosed cancer and a date of diagnosis; and (iii) generating, using the annotated medical records, the curated cancer dictionary, comprising a plurality of cancer-related terms each associated with one or more types of cancer.

According to an embodiment, the plurality of medical records and/or the plurality of subjects of the training dataset are curated by a clinician before training the cancer diagnosis model.

According to an embodiment, the cancer diagnosis model is a gradient boosting classifier.

According to an embodiment, the classifier is a gradient boosting classifier.

According to an embodiment, the trained cancer diagnosis model is unable to identify a date of diagnosis, and the generated cancer diagnosis provided by the user interface further indicates that a date of diagnosis could not be identified.

According to another aspect is a cancer diagnosis system. The cancer diagnosis system comprises: an electronic medical record database comprising a plurality of medical records for each of a plurality of cancer patients; a trained cancer diagnosis model configured to generate a cancer diagnosis for the subject, wherein the cancer diagnosis comprises both an identification of a cancer type and a date of diagnosis, and wherein the trained cancer diagnosis model is trained by: (i) providing a curated cancer dictionary, the curated cancer dictionary comprising a plurality of cancer-related terms each associated with one or more types of cancer, wherein each of the plurality of terms in the curated cancer dictionary is associated with one or more of the following plurality of diagnosis categories: histology, diagnosis test method, stage, grade, and invasiveness; (ii) receiving a training dataset, comprising a plurality of medical records for each of a plurality of subjects; (iii) parsing, using the curated cancer dictionary and a natural language processing (NLP) algorithm, the training dataset to identify cancer-related terms in the plurality of medical records, wherein each of the identified cancer-related terms is associated with one or more of the plurality of diagnosis categories; (iv) analyzing, using a classifier, the training dataset to identify a cancer diagnosis date for each of the plurality of subjects; (v) generating a table of parsed cancer-related terms and cancer diagnosis date for each of the plurality of subjects; (vi) training, using the generated tables, a cancer diagnosis model to determine a cancer diagnosis and a cancer diagnosis date for a subject using a plurality of medical health records for that subject; and (vii) storing the trained cancer diagnosis model; a processor configured to: (i) receive, from the medical record database, a plurality of medical records for a subject; (ii) analyze, by the trained cancer diagnosis model, the received plurality of medical records for the subject; and (iii) generate, from the analysis, a cancer diagnosis for the subject; and a user interface configured to provide the generated cancer diagnosis.

According to an embodiment, the processor is further configured to determine, based on the generated cancer diagnosis, a cancer-specific treatment for the subject, wherein the cancer-specific treatment is one or more of radiation therapy, chemotherapy, immunotherapy, and surgery.

According to an embodiment, the cancer-specific treatment is administered to the subject.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a method for generating a cancer diagnosis for a subject, in accordance with an embodiment.

FIG. 2 is a schematic representation of a cancer diagnosis system, in accordance with an embodiment.

FIG. 3 is a flowchart of a method for training a cancer diagnosis model, in accordance with an embodiment.

FIG. 4 is a flowchart of a method for generating the curated cancer dictionary, in accordance with an embodiment.

FIG. 5 is a flowchart of a method for diagnosing a subject with cancer using a cancer diagnosis system, in accordance with an embodiment.

FIG. 6 is an example of sentence-level concept tagging using an NLP algorithm, in accordance with an embodiment.

FIG. 7 is an example visualization of a sentence tagging and model training, in accordance with an embodiment.

FIG. 8A is a graph of a precision score of the model classifiers on identifying diagnoses of various cancers, in accordance with an embodiment.

FIG. 8B is a graph of a recall score of the model classifiers on identifying diagnoses of various cancers, in accordance with an embodiment.

FIG. 8C is a graph of accuracy of the model classifiers on identifying diagnoses of various cancers, in accordance with an embodiment.

FIG. 9 is a graph of prediction accuracy of the model classifiers on identifying diagnosis dates of various cancers, in accordance with an embodiment.

### DETAILED DESCRIPTION OF EMBODIMENTS

The present disclosure describes various embodiments of a cancer diagnosis system and method configured to generate a cancer diagnosis and diagnosis date for a subject using a trained cancer diagnosis model. More generally, Applicant has recognized and appreciated that it would be beneficial to provide an improved cancer diagnosis system and method with increased accuracy. A cancer diagnosis system receives a plurality of medical records for the subject from an electronic health record database. The system uses a trained cancer diagnosis model to analyze the received plurality of medical records for the subject. The trained cancer diagnosis model is trained by: (i) providing a curated cancer dictionary, the curated cancer dictionary comprising a plurality of cancer-related terms each associated with one or more types of cancer, wherein each of the plurality of terms in the curated cancer dictionary is associated with one or more of the following plurality of diagnosis categories: histology, diagnosis test method, stage, grade, and invasiveness; (ii) receiving a training dataset, comprising a plurality of medical records for each of a plurality of subjects; (iii) parsing, using the curated cancer dictionary and a natural language processing (NLP) algorithm, the training dataset to identify cancer-related terms in the plurality of medical records, wherein each of the identified cancer-related terms is associated with one or more of the plurality of diagnosis categories; (iv) analyzing, using a classifier, the training dataset to identify a cancer diagnosis date for each of the plurality of subjects; (v) generating a table of parsed cancer-related terms and cancer diagnosis date for each of the plurality of subjects; (vi) training, using the generated tables, a cancer diagnosis model to determine a cancer diagnosis and a cancer diagnosis date for a subject using a plurality of medical health records for that subject; and (vii) storing the trained cancer diagnosis model. Based on the analysis, the trained cancer diagnosis model generates a cancer diagnosis for the subject, wherein the cancer diagnosis comprises both an identification of a cancer type and a date of diagnosis. The system then provides the generated cancer diagnosis to a user or clinician via a user interface. According to an embodiment, the method further includes determining, based on the generated cancer diagnosis, a cancer-specific treatment for the subject. The clinician can then administer the cancer-specific treatment to the subject, wherein the cancer-specific treatment is one or more of radiation therapy, chemotherapy, immunotherapy, and surgery.

According to an embodiment, the cancer diagnosis systems and methods described or otherwise envisioned herein comprise a machine learning (ML)-based approach for cancer case detection and date of diagnosis (DDx) annotation. The methods and systems apply a new pipeline which maps diagnosis (Dx)-related concepts in EHR data and identifies diagnosed cases and associated DDx using ML models. This method significantly outperforms the traditional ICD code centered method. The methods and systems apply a natural language processing (NLP) tool, such as ConceptMapper, to capture cancer-related entities and parse the sentences. Then a gradient boosting classifier (GBC) is used to detect cases with cancer diagnosis. Following by the case detection, the entity associated temporal information is captured and used to train another GBC-based model to elect DDx of cancer for each identified patient. This cancer diagnosis system and method includes the detection of numerous different common cancer types. According to one embodiment, in order to ensure the quality of the work, the rule for annotating reference standard dataset was evaluated, defined, and refined by human experts.

The embodiments and implementations disclosed or otherwise envisioned herein can be utilized with any patient care system, including but not limited to clinical decision support tools, among other systems. However, the disclosure is not limited to clinical decision support tools, and thus the embodiments disclosed or otherwise envisioned herein can encompass any device or system capable of generating and reporting cancer diagnosis information for a patient.

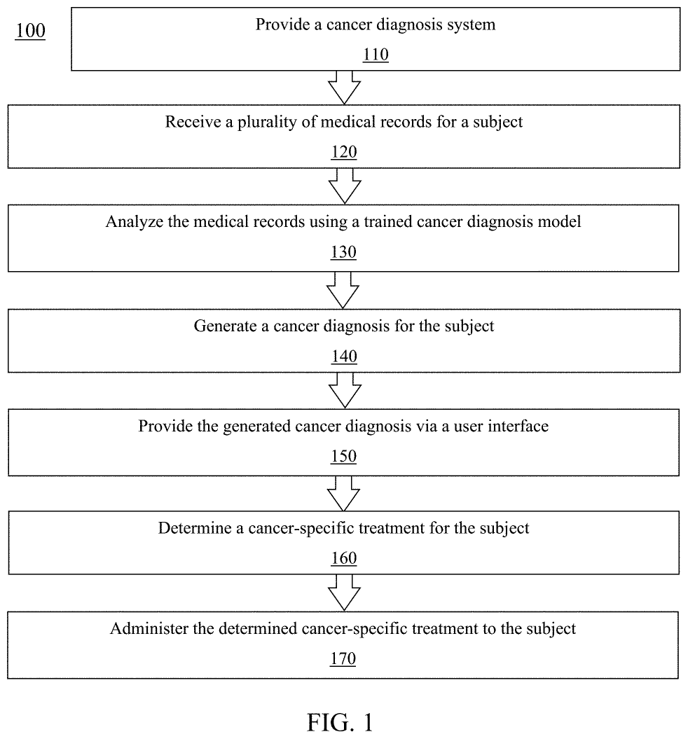

Referring to FIG. 1, in one embodiment, is a flowchart of a method 100 for diagnosing a subject with cancer using a cancer diagnosis system. The methods described in connection with the figures are provided as examples only, and shall be understood not to limit the scope of the disclosure. The cancer diagnosis system can be any of the systems described or otherwise envisioned herein. The cancer diagnosis system can be a single system or multiple different systems.

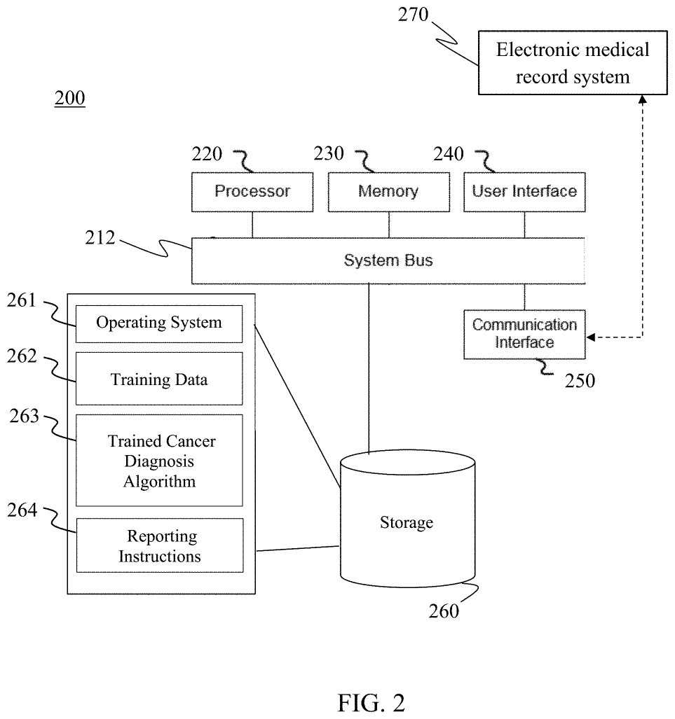

At step 110 of the method, a cancer diagnosis system is provided. Referring to an embodiment of a cancer diagnosis system 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the system 200 may be different and more complex than illustrated. Additionally, cancer diagnosis system 200 can be any of the systems described or otherwise envisioned herein. Other elements and components of the cancer diagnosis system 200 are disclosed and/or envisioned elsewhere herein.

At step 120 of the method, the cancer diagnosis system receives information about a patient. The patient information can be any information about the patient that the trained cancer diagnosis model can or may utilize for analysis as described or otherwise envisioned herein. According to an embodiment, the patient information comprises a plurality of medical records for the subject. The medical records can be, for example, one or more of demographic information about the patient, a diagnosis for the patient, medical history of the patient, and/or any other information. For example, demographic information may comprise information about the patient such as name, age, body mass index (BMI), and any other demographic information. The diagnosis for the patient may be any information about a medical diagnosis for the patient, including both historical and/or current. The medical history of the patient may be any historical admittance or discharge information, historical treatment information, historical diagnosis information, historical exam or imaging information, and/or any other information.

The patient information is received from one or a plurality of different sources. According to an embodiment, the patient information is received from, retrieved from, or otherwise obtained from an electronic medical record (EMR) database or system 270. The EMR database or system may be local or remote. The EMR database or system may be a component of the cancer diagnosis system, or may be in local and/or remote communication with the cancer diagnosis system. The received patient information may be utilized immediately, or may be stored in local or remote storage for use in further steps of the method.

At step 130 of the method, the received patient information is analyzed by a trained cancer diagnosis model of the cancer diagnosis system. The trained cancer diagnosis model can be any model, machine learning algorithm, classifier, or other algorithm capable of analyzing patient information to generate a cancer diagnosis. According to an embodiment, the trained cancer diagnosis model is trained to generate, as one or more elements of the cancer diagnosis, an identification of a cancer type and a date of diagnosis.

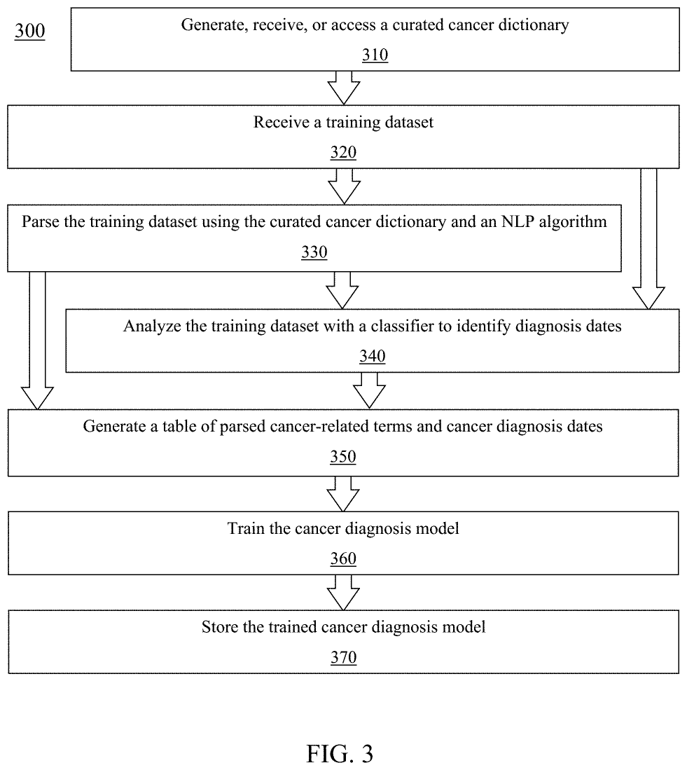

The cancer diagnosis model can be trained by a variety of mechanisms. Referring to FIG. 3, in one embodiment, is a method for training a cancer diagnosis model. The cancer diagnosis model may be trained by the cancer diagnosis system, or may be trained by another system and utilized by the cancer diagnosis system. The trained cancer diagnosis model may be a component of or local to the cancer diagnosis system, or may be remote to the system and accessed and utilized by the system remotely.

At step 310 of the method, a curated cancer dictionary is created or provided. According to an embodiment, the curated cancer dictionary comprises a plurality of cancer-related terms each associated with one or more types of cancer. According to an embodiment, each of the plurality of cancer-related terms in the curated cancer dictionary is associated with one or more of the following plurality of diagnosis categories: histology, diagnosis test method, stage, grade, and invasiveness, although other categories are possible. The received curated cancer dictionary may be utilized immediately, or may be stored in local or remote storage for use in further steps of the method.

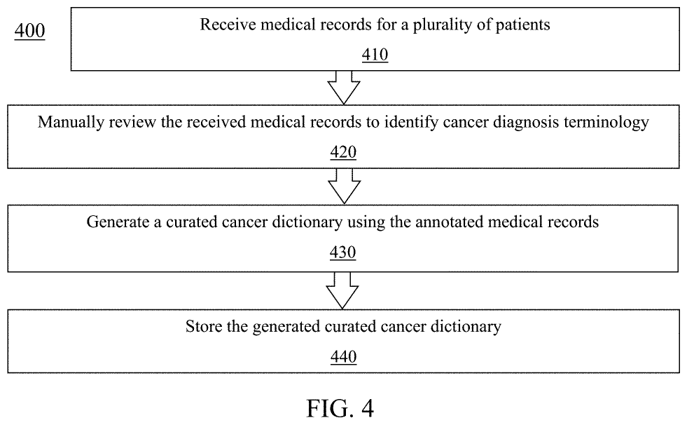

The curated cancer dictionary can be generated by a variety of mechanisms. Referring to FIG. 4, in one embodiment, is a method for generating the curated cancer dictionary. The curated cancer dictionary can be a component of the cancer diagnosis system, or may be in local and/or remote communication with the cancer diagnosis system. At step 410 of the method, a plurality of medical records for each of a plurality of patients is received. The medical records may be received from one or a plurality of different sources. According to an embodiment, the plurality of medical records is received from, retrieved from, or otherwise obtained from an electronic medical record (EMR) database or system 270. The EMR database or system may be local or remote. The EMR database or system may be a component of the cancer diagnosis system, or may be in local and/or remote communication with the cancer diagnosis system. The received plurality of medical records may be utilized immediately, or may be stored in local or remote storage for use in further steps of the method. According to an embodiment, the plurality of patients may be a randomly selected subset of a larger plurality of patients within a database.

At step 420 of the method, the plurality of medical records for each of the plurality of patients is manually reviewed by one or more clinicians or other specialists. According to an embodiment, manual review by the clinician comprises annotating the plurality of medical records with a diagnosed cancer, terms relevant to the cancer type or diagnosis, and/or a date of diagnosis.

At step 430 of the method, the annotated medical records are utilized to generate a curated cancer dictionary. According to an embodiment, the curated cancer dictionary comprises a plurality of cancer-related terms each associated with one or more types of cancer. According to an embodiment, the generated curated cancer dictionary may be refined or otherwise further analyzed or processed to improve or further curate the dictionary.

At step 440 of the method, the generated curated cancer dictionary is stored in memory or a database for subsequent use. The database may be a local and/or remote database. For example, the cancer diagnosis system may comprise a database with the generated curated cancer dictionary.

Returning to method 300 in FIG. 3, at step 320 of the method, the system receives a training dataset. The training dataset comprises patient information which can be any information about the patient that the system can utilize to train the cancer diagnosis model as described or otherwise envisioned herein. At a minimum, the training dataset comprises a plurality of medical records for each of a plurality of subjects. According to an embodiment, the patient information comprises a plurality of medical records for the subject. The medical records can be, for example, one or more of demographic information about the patient, a diagnosis for the patient, medical history of the patient, and/or any other information. For example, demographic information may comprise information about the patient such as name, age, body mass index (BMI), and any other demographic information. The diagnosis for the patient may be any information about a medical diagnosis for the patient, including both historical and/or current. The medical history of the patient may be any historical admittance or discharge information, historical treatment information, historical diagnosis information, historical exam or imaging information, and/or any other information.

The patient information is received, accessed, or retrieved from one or a plurality of different sources. According to an embodiment, the patient information is received from, retrieved from, or otherwise obtained from an electronic medical record (EMR) database or system 270. The EMR database or system may be local or remote. The EMR database or system may be a component of the cancer diagnosis system, or may be in local and/or remote communication with the cancer diagnosis system. The received patient information may be utilized immediately, or may be stored in local or remote storage for use in further steps of the method.

At step 330 of the method, the system parses the training dataset using the curated cancer dictionary and a natural language processing (NLP) algorithm. According to an embodiment, the parsing comprises identifying, using the NLP algorithm based on the curated cancer dictionary, cancer-related terms in the plurality of medical records. According to an embodiment, each of the identified cancer-related terms is associated with one or more of the plurality of diagnosis categories.

The NLP algorithm can be any algorithm capable of analyzing a variety of different types of medical records, such as medical records primarily comprising text such as notes or summary or dictation, for example, from a clinician or specialist or another algorithm. According to an embodiment, the NLP algorithm is an open-source NLP tool such as ConceptMapper, although many other algorithms are possible.

According to an embodiment, the parsed cancer-related terms from the plurality of medical records can be utilized immediately, or may be stored in local or remote storage for use in further steps of the method.

At step 340 of the method, a classifier and the curated cancer dictionary is utilized to analyze the training dataset to identify, from the medical records, a cancer diagnosis date for each of the plurality of subjects. According to an embodiment, the classifier can be any algorithm capable of analyzing a variety of different types of medical records, such as medical records primarily comprising text, to identify a cancer diagnosis date. According to an embodiment, the classifier is a gradient boosting classifier (GBC) trained to identify or elect a DDx for each identified patient. According to an embodiment, the identified or elected cancer diagnosis dates can be utilized immediately, or may be stored in local or remote storage for use in further steps of the method.

At step 350 of the method, the system generates a summary of the parsed cancer-related terms and cancer diagnosis date for each of the plurality of subjects. For example, according to an embodiment, the summary of the parsed cancer-related terms and cancer diagnosis date for each of the plurality of subjects is a data structure stored in a database of the cancer diagnosis system, such as a table or other data structure. According to an embodiment, each of the parsed cancer-related terms is also associated with one of the categories enumerated or otherwise envisioned herein.

At step 360 of the method, the cancer diagnosis model is trained with the generated summary of the parsed cancer-related terms and cancer diagnosis date for each of the plurality of subjects as some or all of the training input. The model is trained to utilize the input and to generate, for each subject, a cancer diagnosis and a cancer diagnosis date. The cancer diagnosis model can be any algorithm capable of being trained using the provided input, and capable of being trained to generate a cancer diagnosis and a cancer diagnosis date for a patient. The cancer diagnosis model can be any classifier, machine learning algorithm, or any other algorithm.

At step 370 of the method, the trained cancer diagnosis model is stored in memory for subsequent analysis. The memory may be local or remote storage, and may be a component of the cancer diagnosis system.

Accordingly, the output of this embodiment of method 300 in FIG. 3 is a trained cancer diagnosis model that can utilize medical records about a subject in order to determine a cancer diagnosis and a date of diagnosis for that subject.

At any step of methods 300 and 400, the cancer diagnosis system may utilize a data pre-processor or similar component or algorithm configured to process received medical records and/or generated training data. For example, the data pre-processor analyzes the received medical records and/or generated training data to remove noise, bias, errors, and other potential issues. The data pre-processor may also analyze the input data to remove low-quality data. Many other forms of data pre-processing or data point identification and/or extraction are possible.

Returning to method 100 in FIG. 1, at step 140 of the method, the cancer diagnosis system generates, from the analysis by the trained cancer diagnosis model, a cancer diagnosis for the subject. According to an embodiment, the cancer diagnosis comprises both an identification of a cancer type and a date of diagnosis. According to an embodiment, the cancer diagnosis can be utilized immediately, or may be stored in local or remote storage for use in further steps of the method.

At step 150 of the method, the cancer diagnosis system provides, via a user interface, the generated cancer diagnosis. According to an embodiment, the provided cancer diagnosis can comprise both an identification of a cancer type and a date of diagnosis. For example, a text-based output or visual representation may be displayed to a medical professional or other user, including the patient, via the user interface of the system. The generated cancer diagnosis may be provided to a user via any mechanism for display, visualization, or otherwise providing information via a user interface. According to an embodiment, the information may be communicated by wired and/or wireless communication to a user interface and/or to another device. For example, the system may communicate the information to a mobile phone, computer, laptop, wearable device, and/or any other device configured to allow display and/or other communication of the report. The user interface can be any device or system that allows information to be conveyed and/or received, and may include a display, a mouse, and/or a keyboard for receiving user commands. As just one non-limiting example, the user interface may be a component of a patient monitoring system.

At step 160 of the method, a clinician utilizes the provided cancer diagnosis to determine a cancer-specific treatment for the subject. According to an embodiment, the provided cancer diagnosis comprises both an identification of a cancer type and a date of diagnosis. Based on one or both of these, as well as their own experience and/or analysis by another algorithm or system, the clinician can determine the most appropriate cancer-specific treatment or treatments for the subject. The most appropriate cancer-specific treatment or treatments can be any treatment intended to address one or more aspects of the diagnosed cancer or side effects of the diagnosed cancer. According to an embodiment, the cancer-specific treatment is one or more of radiation therapy, chemotherapy, immunotherapy, and surgery, among other possible cancer-specific treatment.

At step 170 of the method, a clinician administers the determined cancer-specific treatment to the subject. The determined cancer-specific treatment can be administered to the subject using any method for administering treatment, including but not limited to radiation therapy, chemotherapy, immunotherapy, and surgery, among other possible cancer-specific treatment.

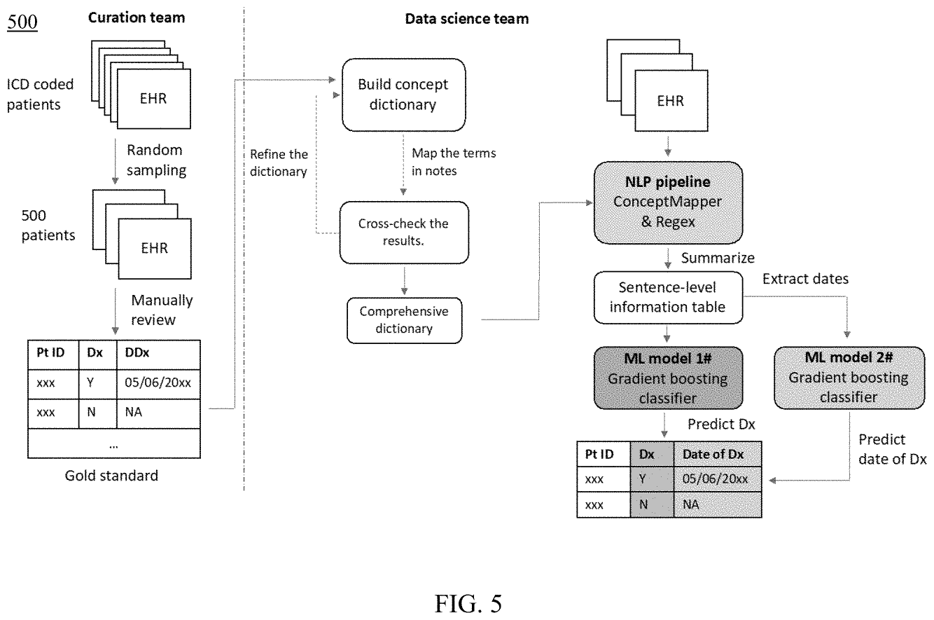

Referring to FIG. 5, in one embodiment, is a flowchart 500 of a method for diagnosing a subject with cancer using a cancer diagnosis system. The methods described in connection with the figure are provided as examples only, and shall be understood not to limit the scope of the disclosure. The cancer diagnosis system can be any of the systems described or otherwise envisioned herein. The cancer diagnosis system can be a single system or multiple different systems.

### Example

Described below is an example of one possible application of the methods and systems described or otherwise envisioned herein. The example is provided only as a possible embodiment of the methods and systems described or otherwise envisioned herein, and is therefore does not limit or prohibit other possible variations and embodiments.

Methods

Sentence-Level Entity Mapping and Parsing

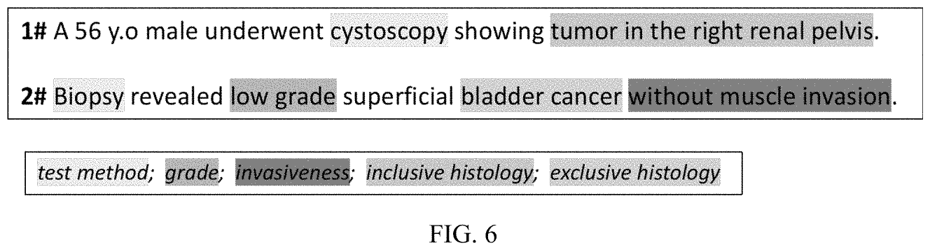

An NLP-based pipeline was applied to EHR data to perform a sentence-level entity mapping and parsing. According to this embodiment, ConceptMapper, an open-source NLP tool, was first applied—along with a comprehensive dictionary of entity terms—to the EHR data (comprising notes and other data) in order to capture the disease-related entities. The comprehensive dictionary consisted of terms that were either inclusively or exclusively associated with the histology, diagnosis test method, stage, grade, invasiveness properties and/or other disease-specific events (e.g., infections of Epstein-Barr virus in candidate cases of Hodgkin lymphoma, among many other examples) of the specific cancer types. After the entity mapping, the mapped terms were then summarized in distinct sentences. Referring to FIG. 6, various disease-related terms were tagged in two example sentences of a non-bladder gastrointestinal origin urothelial cancer (GUTC) patient (1 #) and a bladder cancer patient (2 #), respectively. The mapped histology entities were further classified into sub-categories of inclusive, exclusive, and undetermined histology terms. The inclusive and exclusive histology terms are unambiguous entity statements that could confirm or rule out the diagnosis of corresponding cancer type with high confidence, respectively. As the examples shown in FIG. 6, “tumor in the right renal pelvis” is tagged as an inclusive histology term for GUTC case identification but “bladder cancer” is considered as exclusive histology term to rule out the diagnosis of GUTC. Considering the fact that some terms are not as specific as the histology statement, they might still exist in many irrelevant narrative contexts and be mapped as false positive elements. To eliminate such noise, these tagged sentences were therefore cleaned before passing to the statistical analysis. According to this embodiment, the open-source FastContext module was compiled with the pipeline to analyze the contextuality of the surrounding statement of mapped concepts. According to this embodiment, the concepts in a hypothetical, negated and non-patient statement were omitted. Unreliable sentences that only contained terms of grade, stage, and invasiveness properties, without histology terms, were also removed from the output.

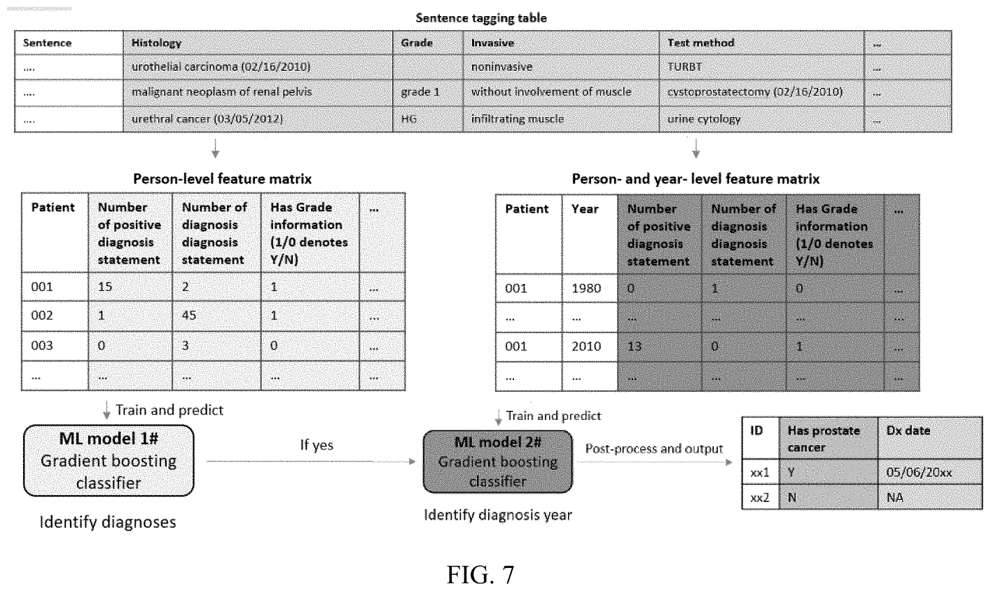

Referring to FIG. 7, in one embodiment, is a visualization of a sentence tagging table generated as described above, which can be utilized to generate a person-level feature matrix and a person- and year-level feature matrix. The person-level feature matrix, which can include a plurality of patients (e.g., Patient 001, Patient 002 . . . Patient 00n) can be utilized to train the gradient boosting classifier (GBC)-based machine learning (ML) model (“ML model 1 #”), and the person- and year-level feature matrix can be utilized to train the other gradient boosting classifier (GBC)-based machine learning (ML) model (“ML model 2 #”). The output of the process may comprise, for example, an output for one or more of the patients with a cancer diagnosis (Y or N indicating Yes or No, although many other indicators are possible) and a Dx date if available. Many other reporting options are possible.

Case Identification

According to this embodiment, a gradient boosting classifier (GBC)-based machine learning (ML) model (“ML model 1 #” in FIG. 5) was trained to identify cases with cancer diagnosis. For each cancer type, a histology-centered feature set was selected and built from the sentence-level entity mapping results. In general, the tagging profile of sentences with histology terms were analyzed. Sentences with inclusive, exclusive, or undetermined histology terms were counted as features for model training. Other than the coarsely categorized features, the tagged information of staging, grade, invasiveness, test method, and disease-specific events in the sentences of histology were further utilized to categorize the records with finer resolution and generate extra features. The GBC model was trained and evaluated under 5-fold cross-validation. During model training, samples of different classes were assigned with different sample weights to eliminate the effect class imbalance. The sample weight of a class is a reciprocal of its sample size percentage to the whole sample population.

DDx Extraction

According to this embodiment, the dates of diagnosis (DDx) were also identified by the pipeline. For the progress notes, the date values were extracted from the diagnosis related sentences via rule-based pattern matching. If multiple dates values were captured from a same sentence, the dates value which was closest to the most important histology terms were used. For the diagnosis related sentences from pathology notes and radiology notes, the sample collection dates and imaging test operation dates were extracted and paired with the records, respectively. Then the extracted date with out-of-range year values (e.g., year value ≤1980 or ≥2021) were removed. For each patient, the extracted dates were eventually summarized in a dated matrix along years from 1980 to 2021. Each row documented the temporal information of the patient in a specific year and was used as a feature vector to train another GBC-based ML model. According to this embodiment, the model was trained to identify the correct year of Dx out of all options for each patient. The year option with highest class log-probability can be elected as the year of Dx. According to one embodiment, if none of the year option had a class log-probability higher than 0.7, no DDx may be reported for such patient. Once the year of Dx was determined, the earliest month and day values can be selected out of the records to stitch the complete DDx results.

ICD9 and ICD10 Results

Among the patients with ICD coded visits, patients with true positive diagnosis were annotated via chart review of EHR notes. As shown in TABLE 1, the ICD coded visits held various true positive rate (TPR) among different cancer types. Patients with acute lymphoblastic leukemia associated ICD coded visits shows the lowest TPR, which is as low as 25.52%. Almost 90% patients with thyroid cancer, bladder cancer and kidney cancer ICD codes were eventually diagnosed with the corresponding cancer. Overall, the ICD coded visits for hematologic cancers (e.g. Hodgkin lymphoma, acute lymphoblastic leukemia) are less reliable than those for solid cancers.

TABLE 1
True positive rate (TPR) of diagnosis
in patients with ICD coded visits

| Cancer type | True positive rate |
|---|---|
| BLACA | 87.44% |
| GUTC | 47.87% |
| HL | 53.46% |
| CECA | 78.68% |
| UTCA | 79.69% |
| DLBCL | 77.82% |
| OVCA | 70.18% |
| PACA | 72.34% |
| THCA | 89.08% |
| LICA | 72.79% |
| ALL | 25.52% |
| KICA | 87.50% |

Identification of Cancer Diagnoses and Diagnosis Dates Using Structured Information

The case identification of cancer diagnosis using structured information was initially evaluated. In brief, information associated with the ICD coded visits were extracted and used as features to train a GBC-based ML model to identify true cancer diagnosis. Based on the ICD codes and assigned dates of codes, times of visits (Nᵥᵢₛᵢₜ) were first gathered at the patient and disease level. Given the fact that the multiple ICD codes were assigned during a visit, a list of confounding ICD codes were created for different cancer types. The list of confounding ICD codes was manually created based on the existing knowledge. For a specific cancer type, the confounding ICD codes represent diseases that are highly related with the targeted cancer, such as tumors originating at adjacent tissues, and might confound the diagnosis results. To assist the case identification of cancer diagnosis, the times of visits from a list of confounding ICD codes were also counted as an extra feature. Then the times of visit of ICD codes and confounding ICD codes were summarized as features to train a GBC-based ML model. As shown in TABLE 2, the mean accuracies across all cancer types are 69.49% and 68.91% on the whole set (training set+validation set) and validation set, respectively.

TABLE 2
Diagnosis identification performance of model
trained by ICD coded structured features

| | | | | Precision | Recall | Accuracy |
|---|---|---|---|---|---|---|
| Cancer | Precision | Recall | Accuracy | (validation | (validation | (validation |
| type | (whole set) | (whole set) | (whole set) | set) | set) | set) |
| BLACA | 99.01% | 67.59% | 71.06% | 97.75% | 66.96% | 69.36% |
| | (±0.67%) | (±2.71%) | (±1.97%) | (±3.33%) | (±7.34%) | (±7.31%) |
| GUTC | 68.63% | 66.24% | 69.10% | 59.66% | 57.27% | 60.44% |
| | (±3.80%) | (±4.56%) | (±1.41%) | (±10.65%) | (±8.41%) | (±2.51%) |
| HL | 62.56% | 93.96% | 71.01% | 60.32% | 91.42% | 68.21% |
| | (±0.20%) | (±0.65%) | (±0.19%) | (±5.24%) | (±3.53%) | (±2.98%) |
| CECA | 91.79% | 76.73% | 76.18% | 88.58% | 75.27% | 72.79% |
| | (±1.60%) | (±7.86%) | (±4.31%) | (±1.67%) | (±7.41%) | (±4.75%) |
| UTCA | 94.05% | 64.12% | 68.12% | 93.31% | 62.59% | 66.40% |
| | (±1.28%) | (±6.86%) | (±4.73%) | (±4.44%) | (±6.04%) | (±1.86%) |
| DLBCL | 97.32% | 80.10% | 82.78% | 91.03% | 75.94% | 75.98% |
| | (±1.16%) | (±2.09%) | (±1.39%) | (±8.03%) | (±7.21%) | (±7.09%) |
| OVCA | 91.08% | 69.50% | 73.68% | 85.21% | 66.06% | 68.07% |
| | (±2.45%) | (±6.46%) | (±2.63%) | (±9.21%) | (±8.48%) | (±8.72%) |
| PACA | 90.90% | 55.20% | 63.55% | 85.98% | 51.32% | 58.84% |
| | (±1.73%) | (±3.34%) | (±1.55%) | (±3.66%) | (±8.23%) | (±6.00%) |
| THCA | 98.27% | 62.04% | 65.11% | 96.67% | 61.92% | 64.04% |
| | (±1.23%) | (±6.57%) | (±5.02%) | (±3.77%) | (±13.02%) | (±9.65%) |
| LICA | 93.37% | 65.44% | 71.45% | 90.05% | 60.78% | 66.78% |
| | (±0.80%) | (±2.51%) | (±1.47%) | (±4.91%) | (±9.25%) | (±5.81%) |
| ALL | 65.51% | 62.12% | 81.91% | 56.55% | 54.65% | 77.84% |
| | (±3.34%) | (±1.80%) | (±1.08%) | (±5.75%) | (±8.03%) | (±3.03%) |
| KICA | 97.72% | 68.45% | 70.95% | 96.60% | 68.10% | 69.98% |
| | (±0.36%) | (±0.70%) | (±0.69%) | (±2.38%) | (±6.75%) | (±6.11%) |
| AML | 93.11% | 71.87% | 79.54% | 90.09% | 70.36% | 77.14% |
| | (±0.82%) | (±0.61%) | (±0.38%) | (±6.86%) | (±7.41%) | (±3.64%) |

On average, solid cancers held high precision (88.12%) but low recall score (63.38%), due to the intrinsic high TPR of the corresponding ICD coded visits. Because of the same reason, hematologic cancers on average showed low precision score (69.57%) in general. To further improve the identification performance of ICD codes, days of visit was extracted from the structured coded data to make the best use of the structured data. The days of visit (Dᵥᵢₛᵢₜ) information was calculated by the day's span between the first and last ICD coded visit. Considering that the more recent patients may not have enough time to accumulate long day's span, a visit-to-day ratio was determined as well as a normalized feature. The visit-to-day ratio (VDR) can be calculated as shown in the following equation:

$$VDR = \frac{{N}_{visit} - \frac{1}{{{N}_{visit}}^{3}}}{{D}_{visit}} \qquad Eq . 1$$

According to this embodiment, the times of visits per day was determined. An adjustment was applied on the total times of visits to resolve the exception of patients with only 1 ICD coded visit in record. The days of visit and visit-to-day ratio were used as additional features to train another GBC-based ML model, with the previous feature sets. Compared with the previous model, the identification performance of the new ML model was only slightly improved. Most of the cancer types had better prediction accuracies on the whole set, but similar performance on the validation set, indicating an overfit model and limited improvement of the new features on reducing the data perplexity. Only acute lymphoblastic leukemia showed remarkable improvement on the precision, recall and accuracy score of validation set, compared with those from the model trained by times of visit only. The three main features extracted from structured ICD coded record all had significant population distribution overlap between positive and negative cases of cancer diagnosis, across almost all cancer types. Except for cases with acute lymphoblastic leukemia associated ICD codes. Most of the cases with long day's span of acute lymphoblastic leukemia related ICD coded visits were eventually diagnosed. The results have demonstrated that the ICD coded structured information is noisy and contains false positive records. To identify the diagnosis accurately, more information needs to be mined from the unstructured data of EHR.

The extraction of diagnosis date was also evaluated using ICD coded structured information. From the cases with true positive diagnosis of cancer, the ICD and confounding ICD coded visits assigned from year 1980 to year 2021 were counted individually by each year. Then a GBC model was trained to predict the year value associated with cancer diagnosis. As summarized in TABLE 3, the average accuracy of the diagnosis year of cancer is 66.52%. For different cancers, the overall accuracy of diagnosis year identification could range from 42.29% (Hodgkin lymphoma) to 84.57% (pancreatic cancer). The results revealed the unreliability of ICD coded information in EHR data on extracting temporal information of cancer diagnosis.

TABLE 3
Diagnosis date identification performance of model
trained by ICD coded structured features

| Cancer type | Accuracy (whole set) | Accuracy (validation set) | |
|---|---|---|---|
| BLACA | 70.00% (±0.70%) | 68.83% | (±5.63%) |
| GUTC | 75.95% (±0.90%) | 71.53% | (±3.69%) |
| HL | 42.38% (±0.00%) | 42.29% | (±4.45%) |
| CECA | 65.89% (±0.00%) | 65.94% | (±9.39%) |
| UTCA | 62.48% (±0.36%) | 60.75% | (±8.99%) |
| DLBCL | 71.86% (±1.01%) | 66.36% | (±8.28%) |
| OVCA | 56.94% (±0.35%) | 55.34% | (±6.94%) |
| PACA | 86.06% (±0.31%) | 84.57% | (±9.17%) |
| THCA | 58.16% (±0.26%) | 58.05% | (±8.90%) |
| LICA | 81.58% (±0.31%) | 80.19% | (±3.10%) |
| ALL | 67.41% (±0.00%) | 67.39% | (±6.71%) |
| KICA | 68.30% (±0.48%) | 67.08% | (±10.06%) |
| AML | 78.61% (±0.53%) | 76.44% | (±10.39%) |

Identification of Cancer Diagnoses and Diagnosis Dates Using Both Structured and Unstructured Information

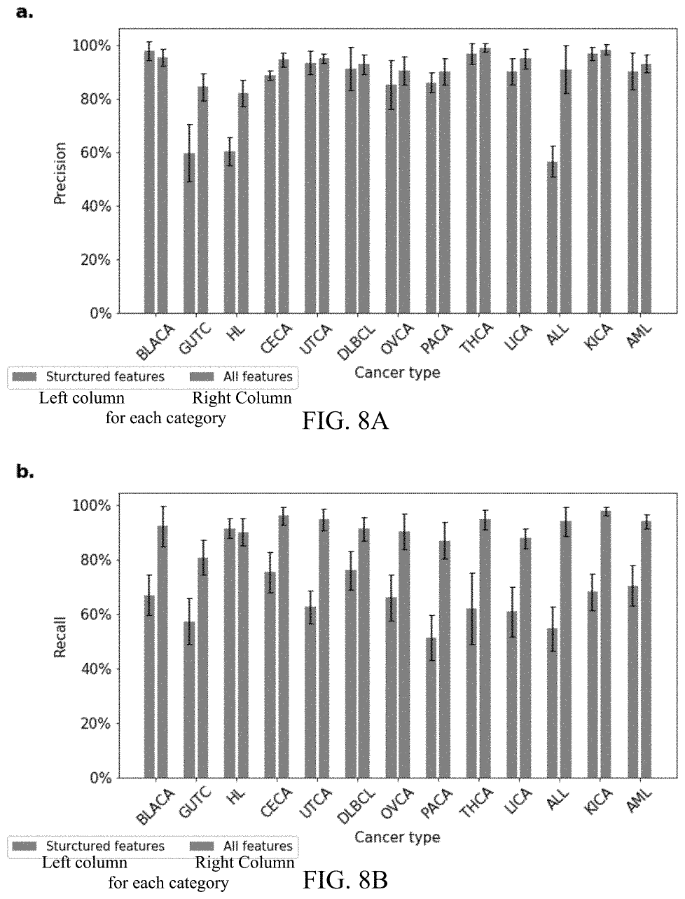

The unstructured information of EHR data was also evaluated. As discussed previously, ConceptMapper was first applied on the EHR records to map cancer-related concepts. Then the mapped concepts were cleaned up and summarized into at sentence-level. For each cancer type, sentence-level records were processed and gathered as a set of histology-centered features. The features extracted from unstructured free text were combined with features of structured coded information and applied to train a GBC-based classifier. As shown in TABLE 4, with the additional unstructured information as features, the trained model accomplished mean accuracies of 96.14% and 89.76% on the whole set and validation set across all cancer types, respectively. As shown in FIGS. 8A-8C (comparing results of models trained by structured information-derived features only with those trained by all information (structured+unstructured)-derived features), the recall and accuracy on the validation set of each individual cancer type were all greatly improved. The precision scores were not significantly improved across various cancer types. This can be explained by the distinct intrinsic class distribution of the sample cohorts. The training and prediction started from patients with ICD-coded visits. For most of the solid cancers, 70%-90% of the patients with ICD-coded visits were found to have true positive diagnosis (TABLE 1), where the negative class will be assigned with higher weight while training the classifier model to avoid underfitting. In this case, if the feature set was not distinguishing enough, introducing false negative cases to keep the accuracy on true negative cases was much favored by the model, which therefore led to high precision score but low recall score.

TABLE 4
Diagnosis identification performance of model trained by
ICD coded structured features and unstructured features.

| | | | | Precision | Recall | Accuracy |
|---|---|---|---|---|---|---|
| Cancer | Precision | Recall | Accuracy | (validation | (validation | (validation |
| type | (whole set) | (whole set) | (whole set) | set) | set) | set) |
| BLACA | 99.06% | 96.09% | 95.78% | 95.36% | 92.14% | 88.95% |
| | (±0.66%) | (±1.65%) | (±1.10%) | (±3.12%) | (±7.33%) | (±4.53%) |
| GUTC | 95.81% | 92.57% | 94.50% | 84.27% | 80.73% | 83.89% |
| | (±0.93%) | (±1.78%) | (±0.84%) | (±5.08%) | (±6.45%) | (±2.45%) |
| HL | 90.40% | 96.73% | 93.69% | 82.00% | 89.96% | 86.17% |
| | (±1.53%) | (±0.75%) | (±0.84%) | (±4.84%) | (±4.99%) | (±3.93%) |
| CECA | 98.78% | 98.22% | 97.65% | 94.46% | 95.94% | 92.28% |
| | (±0.52%) | (±1.01%) | (±0.42%) | (±2.65%) | (±3.14%) | (±1.52%) |
| UTCA | 99.01% | 97.75% | 97.42% | 95.05% | 94.63% | 91.82% |
| | (±0.35%) | (±0.96%) | (±0.81%) | (±1.80%) | (±3.99%) | (±3.11%) |
| DLBCL | 98.55% | 97.68% | 97.07% | 92.68% | 91.12% | 87.60% |
| | (±0.75%) | (±0.79%) | (±0.41%) | (±3.59%) | (±4.44%) | (±2.46%) |
| OVCA | 97.27% | 95.80% | 95.16% | 90.41% | 90.32% | 86.67% |
| | (±0.82%) | (±1.68%) | (±1.15%) | (±5.11%) | (±6.62%) | (±5.63%) |
| PACA | 97.40% | 95.29% | 94.75% | 90.05% | 86.88% | 83.31% |
| | (±0.79%) | (±1.13%) | (±0.92%) | (±5.05%) | (±6.72%) | (±5.90%) |
| THCA | 99.80% | 96.60% | 96.80% | 98.96% | 94.61% | 94.38% |
| | (±0.27%) | (±0.59%) | (±0.49%) | (±1.42%) | (±3.62%) | (±3.60%) |
| LICA | 98.35% | 92.04% | 93.07% | 94.84% | 87.80% | 87.65% |
| | (±0.90%) | (±1.06%) | (±0.54%) | (±3.67%) | (±3.61%) | (±3.22%) |
| ALL | 96.47% | 98.25% | 98.63% | 90.84% | 93.97% | 95.85% |
| | (±1.55%) | (±1.20%) | (±0.45%) | (±8.97%) | (±5.35%) | (±2.43%) |
| KICA | 99.62% | 98.03% | 97.94% | 98.16% | 97.67% | 96.30% |
| | (±0.40%) | (±0.39%) | (±0.29%) | (±1.94%) | (±1.65%) | (±0.90%) |
| AML | 98.31% | 97.29% | 97.31% | 93.02% | 93.94% | 92.00% |
| | (±0.70%) | (±1.01%) | (±0.69%) | (±3.35%) | (±2.60%) | (±2.39%) |

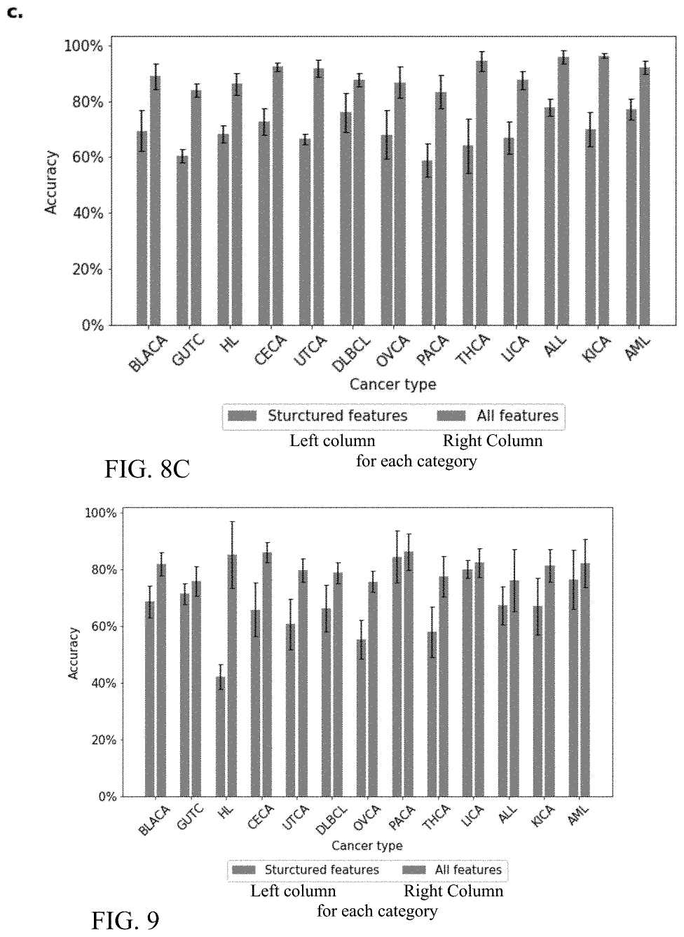

The associated diagnosis dates were also identified from the cases with cancer diagnosis. Similarly, the diagnosis event-related dates were first extracted from sentence-level records. Then the extracted dates were sorted by data sources and counted by each year. Event dates that are out of the range between year 1980 to year 2021 were excluded. A GBC model was trained to predict the year value associated with cancer diagnosis, using the features from both structured and unstructured information. As summarized in TABLE 5 and FIG. 9 (showing prediction accuracy of the model classifiers on identifying diagnosis dates of various cancers, comparing the results of models trained by structured information-derived features only with those trained by all information (structured+unstructured)-derived features), the addition of unstructured information-derived features boosted the identification accuracy of diagnosis dates from 68.13% and 66.52% to 89.43% and 80.78% on whole set and validation set, respectively.

TABLE 5
Diagnosis date identification performance of model trained
by ICD coded structured features and unstructured features.

| Cancer type | Accuracy (whole set) | Accuracy (validation set) | |
|---|---|---|---|
| BLACA | 91.88% (±0.70%) | 82.09% | (±4.15%) |
| GUTC | 86.71% (±1.18%) | 75.95% | (±5.23%) |
| HL | 90.99% (±1.52%) | 85.25% | (±11.88%) |
| CECA | 91.63% (±0.85%) | 86.03% | (±3.52%) |
| UTCA | 88.10% (±0.72%) | 79.76% | (±4.07%) |
| DLBCL | 93.37% (±1.15%) | 78.90% | (±3.76%) |
| OVCA | 85.48% (±0.94%) | 75.79% | (±3.72%) |
| PACA | 92.69% (±1.10%) | 86.29% | (±6.52%) |
| THCA | 85.29% (±0.51%) | 77.60% | (±7.07%) |
| LICA | 93.79% (±1.06%) | 82.49% | (±5.03%) |
| ALL | 82.81% (±1.77%) | 76.26% | (±11.08%) |
| KICA | 87.02% (±0.71%) | 81.44% | (±5.74%) |
| AML | 92.83% (±1.04%) | 82.29% | (±8.57%) |

Referring to FIG. 2 is a schematic representation of a cancer diagnosis system 200. System 200 may be any of the systems described or otherwise envisioned herein, and may comprise any of the components described or otherwise envisioned herein. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, the electronic medical record system 270 is an electronic medical records database from which the information about a plurality of patients, including demographic, diagnosis, and/or treatment information may be obtained or received. According to an embodiment, the electronic medical record system 270 is an electronic medical records database from which the training data utilized to train the cancer diagnosis model. The training data can be any data that will be utilized to train the algorithm. The training data can comprise any other information. The electronic medical records database may be a local or remote database and is in direct and/or indirect communication with system 200. Thus, according to an embodiment, the cancer diagnosis system comprises an electronic medical record database or system 270.

According to an embodiment, storage 260 of system 200 may store one or more algorithms, modules, and/or instructions to carry out one or more functions or steps of the methods described or otherwise envisioned herein. For example, the system may comprise, among other instructions or data, training data 262, a trained cancer diagnosis model or algorithm 263, and/or reporting instructions 264.

According to an embodiment, training data 262 is the initial and/or the modified training data utilized to train and/or retrain the cancer diagnosis model or algorithm 263. The training data can be any data that will be utilized to train or retrain the algorithm. The training data can comprise any other information. According to an embodiment, the training data 262 can additionally and/or alternatively be stored remotely from the system.

According to an embodiment, the cancer diagnosis system comprises a trained cancer diagnosis model or algorithm 263. The trained model can be any algorithm, classifier, or model capable of creating the output, including but not limited to machine learning algorithms, classifiers, and other algorithms. The trained algorithm is a unique algorithm based on the training data used to train the algorithm. Once generated, the retrained algorithm can be utilized or deployed immediately, or it may be stored in local and/or remote memory for future use and/or deployment. Thus, the system comprises a cancer diagnosis model or algorithm 263 configured to generate the cancer diagnosis for a subject as described or otherwise envisioned herein.

According to an embodiment reporting instructions 265 direct the system to generate and provide to a user via a user interface information comprising a cancer diagnosis generated by the cancer diagnosis algorithm. According to an embodiment, the cancer diagnosis comprises both an identification of a cancer type and a diagnosis date. Alternatively, the information may be communicated by wired and/or wireless communication to another device. For example, the system may communicate the information to a mobile phone, computer, laptop, wearable device, and/or any other device configured to allow display and/or other communication of the information.

According to an embodiment, the cancer diagnosis system is configured to process many thousands or millions of datapoints in the input data used to train the cancer diagnosis algorithm, as well as to process and analyze the vast plurality of input data. For example, generating a functional and skilled trained cancer diagnosis algorithm using an automated process such as feature identification and extraction and subsequent training requires processing of millions of datapoints from input data and the generated features. This can require millions or billions of calculations to generate a novel trained cancer diagnosis algorithm from those millions of datapoints and millions or billions of calculations. As a result, each trained cancer diagnosis algorithm is novel and distinct based on the input data and parameters of the machine learning algorithm, and thus improves the functioning of the cancer diagnosis system. Thus, generating a functional and skilled trained cancer diagnosis algorithm comprises a process with a volume of calculation and analysis that a human brain cannot accomplish in a lifetime, or multiple lifetimes.

In addition, the cancer diagnosis system can be configured to continually receive patient data, perform the analysis, and provide periodic or continual updates via the report provided to a user for the patient. This requires the analysis of thousands or millions of datapoints on a continual basis to optimize the reporting, requiring a volume of calculation and analysis that a human brain cannot accomplish in a lifetime. By providing an improved cancer diagnosis for a patient using the cancer diagnosis model as described or otherwise envisioned herein, this novel cancer diagnosis system has an enormous positive effect on patient analysis and care compared to prior art systems.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method (100) for diagnosing a subject with cancer using a cancer diagnosis system (200), comprising:
receiving (120), from an electronic health record database, a plurality of medical records for a subject;
analyzing (130), by a trained cancer diagnosis model (263) of the system, the received plurality of medical records for the subject, wherein the cancer diagnosis model is trained by:
(i) providing (320) a curated cancer dictionary, the curated cancer dictionary comprising a plurality of cancer-related terms each associated with one or more types of cancer, wherein each of the plurality of terms in the curated cancer dictionary is associated with one or more of the following plurality of diagnosis categories: histology, diagnosis test method, stage, grade, and invasiveness;
(ii) receiving (330) a training dataset, comprising a plurality of medical records for each of a plurality of subjects;
(iii) parsing (340), using the curated cancer dictionary and a natural language processing (NLP) algorithm, the training dataset to identify cancer-related terms in the plurality of medical records, wherein each of the identified cancer-related terms is associated with one or more of the plurality of diagnosis categories;
(iv) analyzing (350), using a classifier, the training dataset to identify a cancer diagnosis date for each of the plurality of subjects;
(v) generating (360) a table of parsed cancer-related terms and cancer diagnosis date for each of the plurality of subjects;
(vi) training (370), using the generated tables, a cancer diagnosis model to determine a cancer diagnosis and a cancer diagnosis date for a subject using a plurality of medical health records for that subject; and
(vii) storing (380) the trained cancer diagnosis model;
generating (140), by the analysis, a cancer diagnosis for the subject, wherein the cancer diagnosis comprises both an identification of a cancer type and a date of diagnosis;
providing (150), via a user interface (240) of the system, the generated cancer diagnosis.

2. The method of claim 1, further comprising:
determining (160), based on the generated cancer diagnosis, a cancer-specific treatment for the subject; and
administering (160) the cancer-specific treatment to the subject, wherein the cancer-specific treatment is one or more of radiation therapy, chemotherapy, immunotherapy, and surgery.

3. The method of claim 1, wherein the curated cancer dictionary is generated by: (i) receiving (410) a plurality of medical records for a plurality of patients, wherein the plurality of patients may be a randomly selected subset of a larger plurality of patients; (ii) manually reviewing (420), by a clinician, the plurality of medical records for each of the plurality of patients, wherein manually reviewing by the clinician comprises annotating the plurality of medical records with a diagnosed cancer and a date of diagnosis; and (iii) generating (430), using the annotated medical records, the curated cancer dictionary, comprising a plurality of cancer-related terms each associated with one or more types of cancer.

4. The method of claim 1, wherein the plurality of medical records and/or the plurality of subjects of the training dataset are curated by a clinician before training the cancer diagnosis model.

5. The method of claim 1, wherein the cancer diagnosis model is a gradient boosting classifier.

6. The method of claim 1, wherein the classifier is a gradient boosting classifier.

7. The method of claim 1, wherein the trained cancer diagnosis model is unable to identify a date of diagnosis, and the generated cancer diagnosis provided by the user interface further indicates that a date of diagnosis could not be identified.

8. A cancer diagnosis system (200) configured to diagnose a subject with cancer, comprising:
an electronic medical record database (270) comprising a plurality of medical records for each of a plurality of cancer patients;
a trained cancer diagnosis model (263) configured to generate a cancer diagnosis for the subject, wherein the cancer diagnosis comprises both an identification of a cancer type and a date of diagnosis, and wherein the trained cancer diagnosis model is trained by:
(i) providing (320) a curated cancer dictionary, the curated cancer dictionary comprising a plurality of cancer-related terms each associated with one or more types of cancer, wherein each of the plurality of terms in the curated cancer dictionary is associated with one or more of the following plurality of diagnosis categories: histology, diagnosis test method, stage, grade, and invasiveness;
(ii) receiving (330) a training dataset, comprising a plurality of medical records for each of a plurality of subjects;
(iii) parsing (340), using the curated cancer dictionary and a natural language processing (NLP) algorithm, the training dataset to identify cancer-related terms in the plurality of medical records, wherein each of the identified cancer-related terms is associated with one or more of the plurality of diagnosis categories;
(iv) analyzing (350), using a classifier, the training dataset to identify a cancer diagnosis date for each of the plurality of subjects;
(v) generating (360) a table of parsed cancer-related terms and cancer diagnosis date for each of the plurality of subjects;
(vi) training (370), using the generated tables, a cancer diagnosis model to determine a cancer diagnosis and a cancer diagnosis date for a subject using a plurality of medical health records for that subject; and
(vii) storing (380) the trained cancer diagnosis model;
a processor (220) configured to: (i) receive, from the medical record database, a plurality of medical records for a subject; (ii) analyze, by the trained cancer diagnosis model, the received plurality of medical records for the subject; and (iii) generate, from the analysis, a cancer diagnosis for the subject;
a user interface (240) configured to provide the generated cancer diagnosis.

9. The cancer diagnosis system of claim 8, wherein the processor is further configured to determine, based on the generated cancer diagnosis, a cancer-specific treatment for the subject, wherein the cancer-specific treatment is one or more of radiation therapy, chemotherapy, immunotherapy, and surgery.

10. The cancer diagnosis system of claim 9, wherein the cancer-specific treatment is administered to the subject.

11. The cancer diagnosis system of claim 8, wherein the curated cancer dictionary is generated by: (i) receiving (410) a plurality of medical records for a plurality of patients, wherein the plurality of patients may be a randomly selected subset of a larger plurality of patients; (ii) manually reviewing (420), by a clinician, the plurality of medical records for each of the plurality of patients, wherein manually reviewing by the clinician comprises annotating the plurality of medical records with a diagnosed cancer and a date of diagnosis; and (iii) generating (430), using the annotated medical records, the curated cancer dictionary, comprising a plurality of cancer-related terms each associated with one or more types of cancer.

12. The cancer diagnosis system of claim 8, wherein the plurality of medical records and/or the plurality of subjects of the training dataset are curated by a clinician before training the cancer diagnosis model.

13. The cancer diagnosis system of claim 8, wherein the cancer diagnosis algorithm is a gradient boosting classifier.

14. The cancer diagnosis system of claim 8, wherein the classifier is a gradient boosting classifier.
